(19) United States
(12) Patent Application Publication
(10) Pub. No.: US 2024/0122578 A1
(43) Pub. Date: Apr. 18, 2024

(54) Method for non-invasive real time assessment of cardiovascular blood pressure

(71) Applicants: Alexander Brenner, Haifa (IL); Avraham Lorber, Haifa (IL); Felix Brenner, Haifa (IL)

(72) Inventors: Alexander Brenner, Haifa (IL); Avraham Lorber, Haifa (IL); Felix Brenner, Haifa (IL)

(73) Assignee: PI-Harvest Holding AG, Schaffhausen (CH)

(21) Appl. No.: 18/332,463

(22) Filed: Jul. 26, 2023

Related U.S. Application Data
(62) Division of application No. 16/797,357, filed on Feb. 21, 2020.

(51) Int. Cl. A61B 8/08; A61B 8/00; A61B 8/04; G06T 7/00

(52) U.S. Cl. CPC A61B 8/5223; A61B 8/04; A61B 8/54; A61B 8/56; G06T 7/0012; A61B 5/021; G06T 2207/30048

(57) ABSTRACT

A method of measurement and calculation of intracardiac pressures based on non-invasive medical imaging is presented, wherein the pressure measurements are performed by means of the image stream with further estimation of the volumes of oscillating traceable regions within the heart vicinity. The volume estimates are tied to pressure values in such oscillating traceable regions as left/right atria and ventricles, pulmonary artery and aorta. The invention permits to assess non-invasively and in real time the pressure in any part of the heart and large blood vessels, and calculate the major markers of heart failure, cardiomyopathy, ventricular ischemia, infarction and other heart related diseases.

### PRESENT FIELD

The present invention belongs to the field of medical imaging utilized for non-invasive real time determination of a cardiovascular blood pressure in heart chambers and major blood vessels connected to the heart.

### BACKGROUND

Diseases including congestive heart failure (CHF), abdominal aortic aneurysm (AAA), pulmonary artery hypertension (PAH), are a major cause of premature death. There is a desire to be able to provide an advantageous monitoring of intravascular and/or intracardiac blood pressure, with continuous monitoring. Based on such blood pressure measurements, diagnosis and treatment of users can be based on a unique level, preventing substantial populations from premature death.

Currently, the monitoring of intracardiac blood pressures is possible only during diagnostic catheterization procedure, an invasive process when a pressure meter is physically penetrated into the chamber/artery in question, and is performed under certain medical conditions from time to time.

Implantable sensors used for post-operative continuous monitoring may be introduced during such catheterization procedure and left in the desired heart region. Such invasive measurements of intra-cardiac pressures are desired to be minimized due to complexity of the procedure and related complications and risk.

There are two types of implantable sensors. Active implantable sensors that need a rechargeable energy source, which is related to a number of apparent disadvantages. Passive implantable sensors which are typically electromagnetic, providing an electromagnetic signal when irradiated from the external to the human body source of electromagnetic energy mainly in radio frequencies (RF). These sensors have a considerable drawback due to the locality of their position and ability to measure the pressure only in their circumference, also these sensors have electronics incorporated and have thus related disadvantages, such as issues of biocompatibility, size or reliability of the implanted sensor over time. Moreover, while a part of the RF energy reaches the implanted RF sensor, a considerable amount of the RF energy is absorbed by the body which may cause potential problems in living organisms. Energy transmitted from outside the body may be converted in these implants to power the electronics, make measurements and transmit measurement results back to the outer detecting system. Such detecting system, positioned externally to the human body registers the electromagnetic field radiated by the circuit of the implanted sensor and converts it to readable data.

An example of electromagnetic sensor is described in the U.S. Pat. No. 7,245,117 B1 with the title “Communicating with implanted wireless sensor”, the resonant frequency of a sensor is determined for energizing the system to burst the RF energy at predetermined frequencies and amplitudes. A similar technology is described in the U.S. Pat. No. 8,894,582 B2.

Cardiovascular ultrasound measurements are known, but restricted to either catheter based ultrasound transceivers introduced into the body, or regular and Doppler ultrasound measurements. Blood pressure in peripheral vessels may be measured non-invasively from the outside of the body using ultrasound. However, calibration to actual pressure values of such ultrasound-based methods is complex and not always reliable. Moreover, such methods cannot selectively measure pressure at specific depths and places in the body, e.g. in the aorta or the heart. Other, non-invasive techniques include methods to examine dimensions of blood vessels, or methods based on examining blood flow and are based on Doppler ultrasound or other ultrasound imaging methods, as disclosed, for instance, in U.S. Pat. Nos. 5,411,028, 5,477,858 A, 5,544,656, 6,814,702 B2, 5,724,973 A, US 20140081144 A1, EP 1421905 A1, U.S. Pat. No. 7,128,713 B2, WO 2007087522 A2, US 20080119741 A1, U.S. Pat. No. 7,736,314 B2, US 20130197367 A1, or US 20130006112.

For example in U.S. Pat. No. 5,520,185 A with the title “Method for recognition and reduction of blood speckle in blood vessel imaging system”, a method for enhancing an intravascular ultrasound blood vessel image system is disclosed. It is explained how ultrasound echoes representing vessel walls are distinguished from ultrasound echoes from blood flow by using a classifier which employs the mean and variance of the raw data of greyscale intensities as acquired directly from an ultrasound scanner-detector.

In U.S. Pat. No. 5,800,356 with the title “Ultrasonic diagnostic imaging system with Doppler assisted tracking of tissue motion”, a method for tracing the border of tissue through temporarily acquired scan lines using velocity information corresponding to the tissue edges to trace the denoted border is disclosed.

In U.S. Pat. No. 6,258,031 B1 with the title “Ultrasound diagnostic apparatus”, the velocity of a blood flow and velocity of a blood vessel walls at the same time are measured by an ultrasound with phase detecting.

In US 20090171205 A1 with the title “Method and system for locating blood vessels”, a method utilizing the direct ultrasound sounding for detecting the blood vessels and precisely determining of their depth and diameter is disclosed.

In U.S. Pat. No. 8,469,887 B2 with the title “Method and apparatus for flow parameter imaging”, a method using pulse-wave spectral Doppler imaging allowed to obtain an ultrasound image as a sectional image of the blood vessel, including the inner and outer walls.

Other methods and systems for blood pressure measurements in the blood vessels using Doppler ultrasound imaging are disclosed in: U.S. Pat. No. 5,749,364 A1, WO 20010000 A9, US 20070016037 A1, US 20050015009 A1, US 20140180114 A1, US 20140148702, U.S. Pat. No. 8,968,203 B2, US 20150289836.

In US 20150230774 A1 with the title “Blood pressure monitor and method”, non-invasive continuous real-time monitoring of an arterial blood pressure is disclosed using Doppler probes for systolic and diastolic blood pressure.

In the U.S. Pat. No. 7,404,800B2 a hybrid Left Ventricular End-Diastolic Pressure (LVEDP) monitor is disclosed. The patent refers to unspecified non-invasive pressure measurement devices (“barographs”), yet does not disclose how they produce the “pressure waveform” that is therein used for subsequent analysis and unlike current disclosure does not rely on multi-dimensional image processing.

The above discussed non-invasive ultrasound or Doppler ultrasound methods for the examination of the blood vessels have a number of explicit deficiencies and it is desirous to overcome each of these deficiencies, alone or in combination. Deficiencies include but are not limited to the below:
1. Reproducibility and accuracy of the examination of the blood vessel is highly dependent on the correct orientation of the ultrasound beam's propagation direction (the axis of the ultrasound transducer) relatively to the vessel's longitudinal axis being interrogated. The speed of blood flow is measured by converting of the value of the shift of the Doppler frequency Δf using the Doppler equation:

V=(c×Δf)/(2f₀×cos α),
where V is the velocity of the blood flow, c is the speed of sound in the tissue, f₀ is the initial frequency of the signal, and α is the angle between the direction of the blood flow and the axis of the ultrasound beam. The angle α strongly affects the value of the measured Doppler frequency Δf which in turn is used to calculate of the speed of the organic reflectors in the blood flow.
2. Reliability and precision of blood vessel examination including blood pressure measurement based on ultrasound can be improved. For instance, the Doppler frequency spectra display the blood flow information from a certain area at a given depth, (control volume), and do not provide information about blood flow in other parts of the vessel which are visible on the ultrasound image. Therefore, in case choosing an inadequate control volume (ex., when cos α˜0) all diagnostic information will be incorrect.
3. References to non-specified devices and amplitude sensors (such as in U.S. Pat. No. 7,404,800B2) that produce the intracardiac pressure without further explanations.

Insufficient accuracy of results from hemodynamic measurements in blood vessels using certain Doppler methods are well documented. For example, in: S. B. Coffi, D. Th. Ubbink and D. A. Legemate. Non-invasive Techniques to Detect Subcritical Iliac Artery Stenosis. Eur. J. Vascular and Endovascular Surgery, 29, 2005; Ricardo Cesar, Rocha Moreira. Comparative study of Doppler ultrasonography with arteriography in the evaluation of aortic occlusive disease. Journal Vascular Brasileiro,8, Jan./Mar. 2009; or Vilhelm Schaberle. Ultrasonography in Vascular Diagnosis. A Therapy-Oriented Textbook and Atlas. Second Edition. Springer Heidelberg-Dordrecht-London-New-York, 2011.

The article Gernot Schulte-Altedorneburg, Dirk W. Droste, Szabolcs Felszegny, Monica Kellerman et al., Accuracy in vivo Carotid B-mode Ultrasound Compared with Pathological Analysis: Intima-Media Thickening, Lumen Diameter and Cross-Sectional Area. Stroke: Journal of the American Heart Association, 2001 demonstrates an insufficient accuracy of the results obtained for the examination of blood vessels using of the ultrasound B-mode imaging only.

Several patents are dedicated to using passive sensors placed in the human body and interacting with an external ultrasound source for analysis of physiological parameters of the human organism, as for instance U.S. Pat. Nos. 5,619,997 A, 5,989,190 A, 6,083,165 A, US 20030176789 A1 or US20090171201 A1. However, these devices and methods have a number of drawbacks, namely the following:

1. The disclosures in U.S. Pat. Nos. 5,619,997 A, 5,989,190 A, 6,083,165 A consist in the suggestion that the physical parameters (pressure, temperature, viscosity) defining the state of the medium (including the human body) are determined as a functional relationship P=f(v), where P is the physical parameter and v is the frequency of the ultrasound wave reflected by a passive sensor placed in the medium which is different from the frequency of the primary ultrasound beam due the energy absorption by the sensor.

2. The disclosure in patent application US 20030176789 A1 suggests that the value of a specific physical parameter, such as the pressure, associated with the specific state of any medium (including the human body) is determined as the result of the frequency analysis of the acoustic signal reflected by the passive sensor implanted into the medium. The passive sensor has to be equipped with two parallel to each other reflective surfaces and the reflected signal is the result of the interference of the two acoustic signals: the first signal is reflected by the first reflective surface and second signal reflected by the second reflective surface.

The frequency analysis of the resultant signal permits allocates the frequencies of the maximal attenuation of the intensity and the value of the specific physical parameter is determined on the basis of the correlation relationships between the values of the parameters and the frequencies of the maximum attenuation of the resultant signal. The knowledge of the correlation between the values of the parameters and the frequencies is not sufficient to determine the functional relationship P=F (v). The method is dependent of the frequencies of both the direct and reflected signals, It is desired to provide a more simple method and system that is independent of the frequencies of both the direct and reflected signals which are also present in the following patent: US 20070208293 A1 “Methods and devices for non-invasive pressure measurement in ventricular shunts”. This disclosure relates to a ventricular shunt including a pressure-sensitive body that changes its dimensions in response to the pressure of the cerebrospinal fluid within the shunt.

3. The difference of US 20070208293 A1 from current document lies in several aspects. First, the flow of cerebrospinal fluid is quasi-stationary unlike the turbulent blood flow such as inside the heart chambers which are the subject of the current disclosure. Second, the system from US 20070208293 A1 is tracking the distance changes between the transducer and ultrasonic beam reflecting gas-filled capsule, while in the current description the pressure is determined/estimated as the function of volumes of oscillating traceable regions in a series of images produced by medical imaging device placed fully outside of the body, regardless of presence or absence of any implanted devices. By an oscillating traceable region we mean a region appearing on most images of the series corresponding to the physiological domain where the pressure is measured or calculated, typically one or more of heart chambers, pulmonary artery and/or aorta.

On the other hand, we note the successful approach of the linear regression modeling of the maximal value of the Left Atrium pressure changes through the simultaneous measurements of the Left Atrium pressure with a catheter and trans-esophageal Doppler echocardiography published in the article “Noninvasive assessment of left atrial maximum dP/dt by a combination of transmitral and pulmonary venous flow”, see the Journal of the American College of Cardiology, V. 34, Issue 3, Sept. 1999, P. 795-801, by Satoshi Nakatani, Mario J Garcia, Michael S Firstenberg, Leonardo Rodriguez, Richard A Grimm, Neil L Greenberg, Patrick M McCarthy. However, in this article it had not been reflected that not only Doppler echocardiography but a regular ultrasound or other imaging methods can be used to assess the atrial and more important ventricular (both left and right maximal dP/dt values called Left/Right Ventricular Pressure Rise) blood pressure and not only pressure changes, but absolute pressure values as well. This principle is realized in the present invention.

4. There are several significant differences between the current disclosure and the technology described in the patent US20090171201 A1. The patent US20090171201 A1 claims simultaneous tracking of the pressure via intracardiac pressure meter and the volume via intracardiac echocardiography (ICE). In the current disclosure the simultaneous recording is also used, but only as a part of a preparatory pressure model calibration procedure. As soon as the model is calibrated, it is ready for follow-up pressure calculations based on image processing only.

Additionally, the patent US20090171201 A1 relies on positioning of the ultrasound device within the heart. This requires constructing such a device and performing an invasive procedure to insert it into position through catheterization. Having the ultrasound device positioned inside the heart chamber, and measuring the distances to heart chamber walls it is indeed possible to derive heart chamber volume, however to repeat the measurement, it is necessary to again penetrate the device into the patient's heart chamber, which, again, requires an invasive procedure in hospital conditions.

The idea behind the current invention is to use an ultrasound device positioned outside the body, and rely on data obtained during the calibration procedure, while catheterization is performed on any medical purpose, to estimate the intracardiac pressure as a function of the volume of the appropriate heart chamber.

While in US20090171201 A1 patent it is proposed to simultaneously record ultrasound data (recalculated to the chamber volume assessment) together with pressure data and display them synchronously to the medical team during the catheterization procedure, the proposed method is aimed to use ultrasound data as obtained from a device positioned outside the body paired with pressure data for subsequent estimation of intracardiac pressure using ultrasound device only.

This present disclosure contains amongst others a novel method to calculate and determine said pressure and said method is independent of the frequencies of both the direct and reflected signals. Thus, prior technical solutions, such as disclosed in U.S. Pat. Nos. 5,619,997 A, 5,989,190 A, 6,083,165 A, US 20030176789 or US20090171201 A1 are not analogues both in the methods of data collection and the methods of data processing of the current disclosure.

The approach in the present disclosure is based on the estimation of the pressure as a function of volumes of oscillating traceable regions (e.g. heart chambers) conducted via image processing of ultrasound recordings, or other imaging device technology with similar functionality.

Additionally, the present disclosure contains a provision for utilizing data obtained by calibration procedure from different patients with similar physiological and clinical profiles for increasing performance and accuracy or making the system available for users that did not undergo the calibration procedure.

### SUMMARY OF THE INVENTION

The essence of the current disclosure lies in the description of a direct non-invasive method of measurement of the intracardiac blood pressures and an embodiment apparatus required for its practical implementation.

The apparatus contains a medical imaging device capable of highlighting inner physiological features and streaming image data in real time and is configured to produce an image stream {Jᵢ}_{i=1, . . . N} (where i is the index of the image and N is the number of images in the stream) of cardiovascular movement, and a processor unit (a computer or mobile device) which is configured to receive the said image stream-through a communication protocol and index the images by corresponding time-stamps. The processor unit records, processes the image stream and calculates the blood pressure in the given volume of the cardiovascular structure.

The method provided in the present disclosure is intended for calibration, measurement and calculation of intracardiac blood pressure based on the volumes of cardiovascular structures including but not limited to the Left Atrial Pressure (LAP), Right Atrial Pressure (RAP), Left Ventricular Pressure Rise dP/dt_{max,L} (LVPR), Right Ventricular Pressure Rise dP/dt_{max,R} (RVPR), Pulmonary Artery Pressure (PAP), Left Ventricular Systolic Pressure (LVSP), and Right Ventricular Systolic Pressure (RVSP), Left Ventricular End-Diastolic Pressure (LVEDP) and Right Ventricular End-Diastolic Pressure (RVEDP).

The pressure values within the above positions may provide valuable diagnostic information for potential therapeutical treatment of a user, for example, markers of the right heart failure, cardiomyopathy, right ventricular ischemia and infarction, left heart failure (CHF), myocardial infarction, tamponade, aortic regurgitation and others.

While the change in shape and size of the heart as seen on ultrasound, MM or CT does reflect the changes in intracardiac pressure and the markers for changing of the heart condition can be obtained by the currently proposed method completely non-invasively, the exact value of the pressure is still unknown as it differs from person to person and depends on a variety of physiological factors.

To solve this, we introduce the calibration procedure, which takes advantage of the fact that catheterizations for pressure measurement or other diagnostic or treatment purposes are routinely performed on patients with conditions that make them the primary potential users of the current invention, and subsequently requires only to add a parallel ultrasound recording to any other performed actions in order to obtain two parallel streams of data: ultrasound and pressure. They are compared and a personal formula—a calculation model, which would be valid for the user henceforth is produced. Following the calibration, this model can help the user to measure and detect any changes in the intracardiac pressure in non-invasive way at any place and time of choice and alert the user and connected medical service provider if the changes signal worsening of the health condition.

The present invention relies on a usage of medical imaging devices, such as ultrasound, MM or CT devices, which are capable to produce a time series of images highlighting the boundaries, shape, size and position of inner physiological features such as heart chambers while being positioned fully outside of the body and communicating this series of images in real time to a processor for subsequent analysis.

The series of images (frames) obtained from the imaging device image stream {Jᵢ}_{i=1, . . . N} which hereafter is referred as the T-image, is a 3-dimensional cube of imaging data consisting of individual pixels, where the axes are longitudinal and lateral coordinates of the pixel within the image, the time of frame recording, and the values are brightness and/or color values of the pixel.

The processing software determines the boundaries, shape, size and position of the heart chambers on each of the frames and traces the movement of pixels from image to image to determine their change over time. As the heart beats, the coordinates of the visible boundaries will change to reflect the contraction and expansion, and from the change of those boundaries a change in blood pressure will be derived.

In order to simplify the processing and to enable user-friendly visualization of the T-image, the so-called Characteristic image can be used, which is a form of height-by-time projection where each frame is compressed to a single column. The Characteristic image presents the vertical movement of the heart boundaries. Assuming the imaging device is correctly positioned, the vertical movement will roughly correspond to pressure changes, and its accuracy can be further expanded by more detailed analysis of each of the images as follow from the new notions of Characteristic Image and its derivatives defined below.

Overall, the advantages of the disclosed method over the prior art presented by this disclosure include:
Estimation of intracardiac pressure changes as a function of the observed changes in the size and position of oscillating traceable regions within the vicinity of the heart during non-invasive measurements;
Calibration procedure, which is performed during routine diagnostic catheterization one time per user, and represents a synchronized, simultaneous measurement of intra-cardiac blood pressure with a micro-manometer catheter attached to clinical catheterization pressure measurement monitor coupled with a medical imaging device through a processor unit, which is performing a subsequent image processing analysis and model fit for pressure calculation;
Usage procedure of the imaging device in combination with the processor in order to calculate the intracardiac pressure on obtaining imaging data of the same region in the heart vicinity of the user and utilizing the unique fitted functional parameters of the mathematical model from the calibration procedure to evaluate the imaging data anytime, anywhere, not only for an individual user, but for a class of users with the similar physiological profile.
Process for the analysis of image time series based on the new notions of:
   T-image {Tᵢ}_{i=1, . . . N}, (where i is the index of the image and N is the number of images in the stream) which is defined as a chronological union of said image stream {Jᵢ}_{i=1, . . . N} with corresponding time stamps.
   Characteristic (or Eigen-) Image {Iᵢ}_{i=1, . . . N} of the said T-image {Tᵢ}_{i=1, . . . N}, which is as a chronological union of the averages of the rows or other invariants of the image series {Tᵢ}_{i=1, . . . N} across each given depth, in the way that the first pixel-column I₁ (i=1) of the Characteristic image contains the averages over the rows or other invariants of the first image in time, the second pixel-column I₂ (i=2) of the Characteristic image contains the averages over the rows or other invariants of the second image in time, and finally the last pixel-column I_{N}(i=N) of the Characteristic image contains the averages over the rows or other invariants of the last image in time in the series. Characteristic image method reduces the problem dimensionality while still permits to identify the pressure curve, provides a considerable boost in performance and lowers calculation power requirements which is useful for small or embedded devices.
      The invariants in the Characteristic images can be
         averages of the columns
         vertical or horizontal average gradients
         singular values or eigenvalues of each image packed into the Characteristic image as one matrix
         Fourier, Wavelet or other generalized decomposition images of the Characteristic images defined above.
   Additionally, the Characteristic image represents the dynamics of movement of the oscillating traceable regions through
   T-Image {Tᵢ}_{i=1, . . . N} time derivative streams {Tᵢ}′_{i=1, . . . N}, {Tᵢ}″_{i=1, . . . N}, . . . which are the pixel-wise finite differences of the brightness in greyscale case (or a value of respective color channel in color case): Tᵢ−Tᵢ₋₁ (assuming time difference scale unit is 1). The subsequent averaging along the time scale (mean{Tᵢ}′_{i=1, . . . N}, mean {Tᵢ}″_{i=1, . . . N}) produces an image that shows repeating tissue movement and permits to identify the size and position of the oscillating traceable regions.
The above Process for the analysis of a series of includes:
   Calibration, where the pressure in the target region, acquired from catheterization pressure measurement monitor and corresponding imaging data are both known and the goal is to build a mathematical model of the pressure as a function of imaging data and including the following method steps:
      aligning the directly measured intra-cardiac chamber pressures during catheterization with synchronized imaging data
      processing the image stream {Jᵢ}_{i=1, . . . N} and generate corresponding T-image {Tᵢ}_{i=1, . . . N}
      processing the T-Image {Tᵢ}_{i=1, . . . N} by using time derivative streams {Tᵢ}′_{i=1, . . . N}, {Tᵢ}″_{i=1, . . . N}, . . . or any other convenient method to determine oscillating traceable regions corresponding to cardiovascular structures in heart vicinity and record their shape changes over time,
      creating a set of coordinate parameters {xⱼ}_{j=1, . . . M} (where j is an index of the coordinate and M is the total number of the coordinate parameters) representing the said oscillating traceable region size, form and position at each time corresponding to each frame Tᵢ of the said T-image {Tᵢ}_{i=1, . . . N}, and
      fitting the functional parameters of the mathematical model capable to further calculate the pressure function P inside an oscillating traceable region according to the measured, intra-cardiac chamber pressures during catheterization. The functional parameters of the pressure function P are chosen to perform the best fit to the measured or estimated pressure values Pᵢ=P(tᵢ, {xⱼ}_{j=1, . . . M}ⁱ⊂Tᵢ) of a shape and position of the said oscillating traceable region.
         In case of using T-Image {Tᵢ}_{i=1, . . . N} itself, the function is P(tᵢ)=P(tᵢ, {xⱼ}_{j=1, . . . M}ⁱ ⊂Tᵢ), where xⱼ are a set of coordinate parameters representing the said oscillating traceable region boundaries and position at each time corresponding to each frame Tᵢ of the said T-image {Tᵢ}_{i=1, . . . N}.
         In case of using Characteristic image {Iᵢ}_{i=1, . . . N} of the said T-image {Tᵢ}_{i=1, . . . N}, being a process which simplifies the model reducing its dimension and significantly improves calculation times without significant precision loss, Pᵢ=P(tᵢ, {xⱼ}_{j=1, . . . K}ⁱ ⊂Iᵢ), where {xⱼ}_{j=1, . . . K} are a set of coordinate parameters representing the said oscillating traceable region size and position at each time corresponding to each column Iᵢ of the said Characteristic Image {Iᵢ}_{i=1, . . . N}.
Usage, where the imaging data and the previously acquired mathematical model for calculation of the pressure as a function of imaging data are known, and the goal is to estimate the pressure values using the previously acquired model from the imaging data and including the following method steps:
   processing the image stream {Jᵢ}_{i=, . . . N} and generate corresponding T-image {Tᵢ}_{i=1, . . . N}
   processing the T-Image {Tᵢ}_{i=1, . . . N} by using time derivative streams {Tᵢ}′_{i=1, . . . N}, {Tᵢ}″_{i=1, . . . N}, . . . or any other convenient method to determine oscillating traceable regions corresponding to cardiovascular structures in heart vicinity and record their shape changes over time,
   creating a set of coordinate parameters {xⱼ}_{j=1, . . . M} representing the said oscillating traceable region size, form and position at each time corresponding to each frame Tᵢ of the said T-image {Tᵢ}_{i=1, . . . N}, and
   applying the function Pᵢ=P(tᵢ, {xⱼ}_{j=1, . . . M}ⁱ⊂Tᵢ) produced during the calibration procedure to the above set of coordinate parameters to estimate the pressure values at each time corresponding to each frame Tᵢ of the said T-image {Tᵢ}_{i=1, . . . N} and pressure changes between the frames {ΔP(tᵢ)}_{i=1, . . . N}. In the absence of calibration model for a particular user, machine-learning tools permit to estimate the pressure basing on calibration model from other users with similar physiological parameters.

Accordingly, utilizing the data obtained during calibration procedure, the intracardiac pressure and its dynamic changes within the cardiovascular system can be calculated with high accuracy and stability any time after the calibration procedure when a recording of the calibrated region is provided with the medical imaging device coupled with the processor unit in the framework of the current apparatus.

A collection of fitted mathematical models produced by the calibration procedure on a wide user set, enables to produce generalized mathematical models that may be applied to additional users which have not undergone the calibration procedure during clinical catheterization, but have similar physiological characteristics to those that were calibrated.

### BRIEF DESCRIPTION OF THE DRAWINGS

The below described embodiments with the references to the accompanying drawings present the features and advantages of the current invention. It has to be noted that being an example of a functional system providing the claimed method the following implementation is not limited to mentioned devices/technologies that may be replaced by their similar modalities as long as the said modalities can produce the imaging data and maintain data connections to processor units which, in turn, may be any computing devices restricted only by ability to run processing software and provide necessary data connections and user interfaces:

FIG. 1 depicts a schematic illustration of a blood pressure calibration procedure performed during clinical catheterization. The pressure sensor (101) is located inside the user's heart or major blood vessel in the heart vicinity (102) introduced by catheter (103) through a subclavian jugular or cephalic vein (104). The sensor is connected to pressure monitor (105), which is, in turn, connected to a computer, serving as the processor unit (106). The medical imaging device (107), connected to the processor unit (106) by wired or wireless connection (108), is performing a recording (109) of the user's heart (102) where the sensor (101) is located. The processor unit (106) creates a simultaneous recording from both imaging device (107) and pressure monitor (105), synchronizes the data, performs the calculations and stores them or sends them to remote cloud or other specialized server (110) for storage or performing calculations.

FIG. 2 depicts the typical usage procedure of the pressure measurement method. The imaging device (201), connected by wired or wireless connection (202) to processor unit (203), is pointed towards the user's heart (204), for which the calibration was previously performed and performs a recording (205), sending it to the processor unit. The processor unit either performs the calculations locally or sends the data to remote cloud server or other specialized server (206). The calculations are performed based on previously recorded model and their results are displayed to the user through the UI on the processor unit.

FIG. 3 depicts the typical case of usage procedure in presence of previously recorded calibration model for the user, who performs the measurement procedure (301), sending the data to remote cloud server or other specialized server (302), which retrieves the stored calibration model (303) and calculates the result according to the said model.

FIG. 4 depicts the typical case of usage procedure without previous calibration model for the user. The user performs the recording procedure (401), sending the data to remote cloud server or other specialized server (402), which retrieves the stored calibration models (403) of other users with similar physiological data (age, weight, height, diagnoses, etc.) and uses machine learning to calculate the result according to said models.

FIG. 5 depicts a single frame of imaging data of user's heart (501) as it is received from imaging device.

FIG. 6 depicts two frames of imaging data of user's heart showing the difference between imaging data at different time moments (601, 602).

FIG. 7 depicts the assembled T-Image (701) compiled from imaging data (702) with an appropriate time axis (703).

FIG. 8 depicts two frames of imaging data (801, 802) in different states of the heart movement with detected heart chamber contours (803, 804), and the positions and contours of Right Atrium (805), Left Atrium (806), Right Ventricle (807) and Left Ventricle (808). In the first frame the contour (803) is depicted as formed from a sample set of coordinate points {x 1} (809) that are used during calculation.

FIG. 9 depicts two frames of imaging data (901, 902) with separated contours of Left Ventricle (903, 904) at subsequent time stamps.

FIG. 10 depicts the connection between pressure (1001) and state of heart chamber, in case of the Left Ventricle (1002).

FIG. 11 depicts the process of creation of a Characteristic Image. A frame of imaging data (1101) is compressed (1102) to a single column (1103) using averaging or other invariant method to mark the vertical positions of heart tissue. In the same manner, the T-Image (1104) containing series of frames and a time axis (1105) is compressed to a Characteristic Image (1106) with number of columns identical to number of frames, illustrating the tissue movement over time.

FIG. 12 illustrates the similarity between Characteristic Image (1201) and pressure measurements of LA (1202) and LV (1203) as received from the pressure monitor (105). The LA (1204) and LV (1205) pressure curves are clearly identifiable on the Characteristic Image (1201).

### DESCRIPTION OF EMBODIMENTS

Specific embodiments or examples of the invention will now be described with reference to the accompanying drawings. This invention may, however, can be embodied in many different forms and should not be construed as limited to the embodiments demonstrated herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention.

The present description of the current invention is given with reference to heart chambers and major blood vessels in heart vicinity. It should be born in mind however that the present invention is not limited strictly to heart chambers and major blood vessels, but can be easily adapted to any medium transparent for ultrasound or other waves with the need to measure pressure changes of a liquid flow within a flexible environment.

Alternatively, or in addition to ultrasound, in order to generate series of images to be analyzed for the intracardiac pressure determination, other systems capable of highlighting inner physiological features and streaming image data in real time, for instance Magnetic Resonance Imaging (MRI), or ionizing radiation based imaging systems like Roentgen (X-Ray, Computer tomographic Imaging [CT]) can be provided as medical imaging modalities for generating the input for the pressure determination.

Additionally, while the present description refers to the usage of 2-dimensional cross-section ultrasound imaging of the investigated chamber, as produced by contemporary sensors, the description is not limited to 2-dimensional cross-section ultrasound imaging and can utilize different modalities that produce several 2-dimensional cross-sections or a full 3-dimensional representation of the investigated chamber.

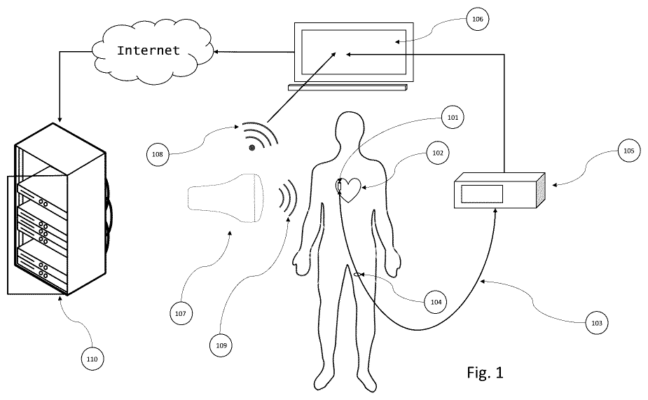

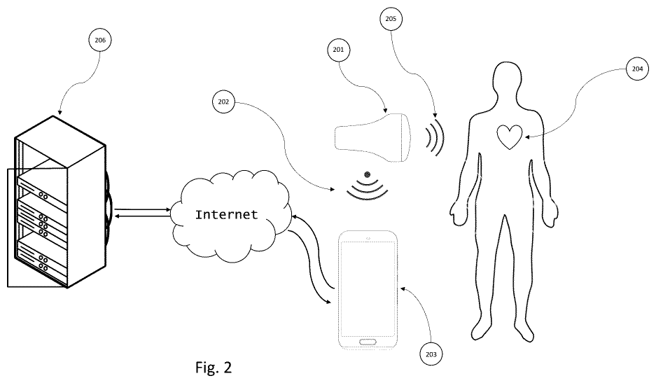

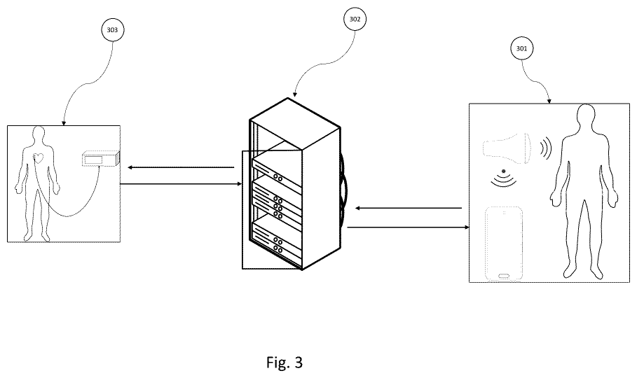

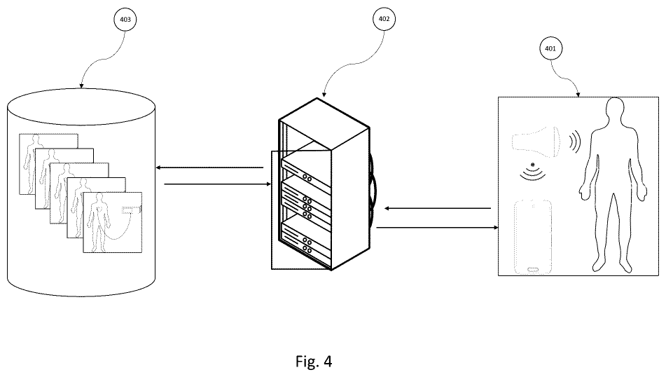

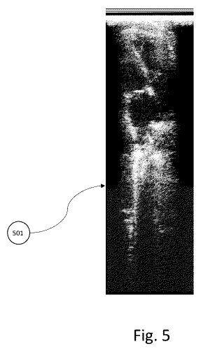

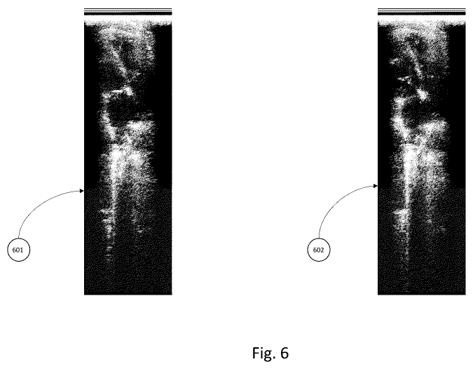

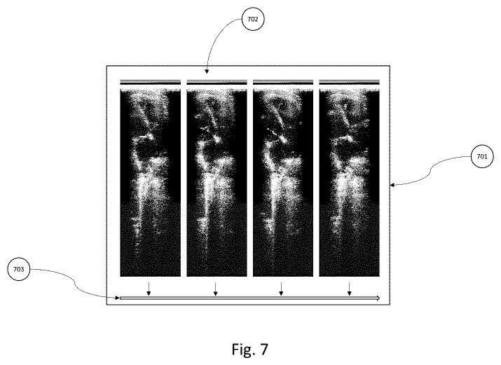

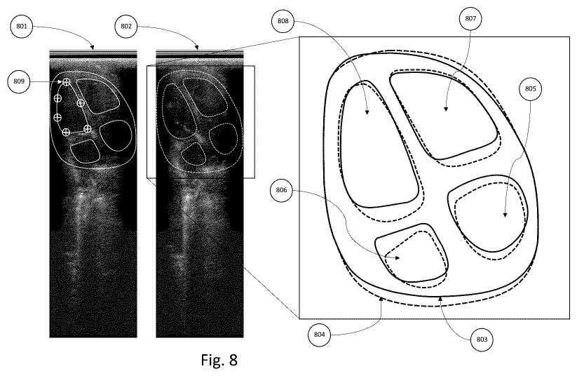

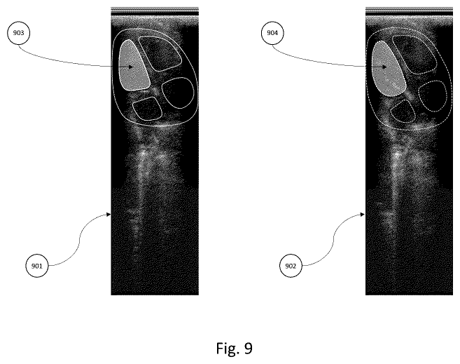

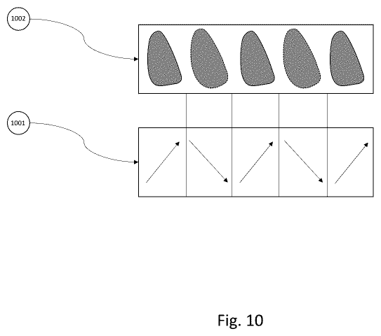

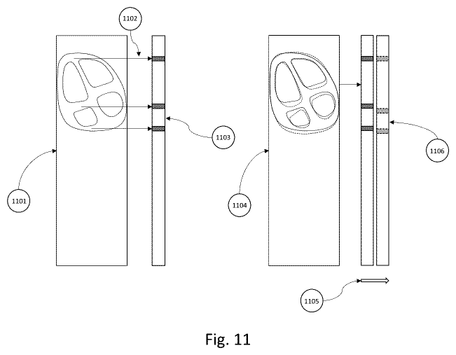

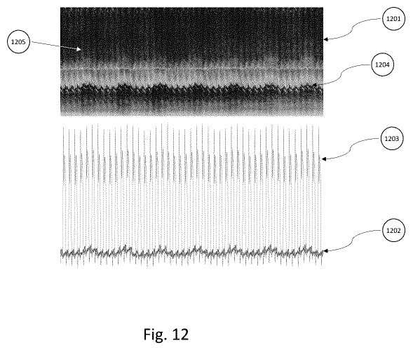

In accordance with the preferred embodiments the hardware system needed to provide the proposed method comprises:
1) For the Calibration Procedure(FIG. 1)
   a) At least one catheter-based blood pressure sensor (101) with analogue or digital data output connected to a standard clinical catheterization pressure measurement monitor (105 medical). The said sensor is located inside one of the user's heart chambers or surrounding blood vessels, such as pulmonary artery, (102) introduced, for example, by catheter (103) through a subclavian jugular or cephalic vein (104). It is assumed that since the calibration procedure is performed during routine scheduled clinical catheterization of the user, the sensor may be already introduced into the problematic region as a part of a procedure, then only its connection to the processor unit as described in items b) and d) below is required.
   b) Medical pressure measurement monitor with digital data output permitting to receive the pressure data from the pressure sensors (101) and stream the output data into the processor unit, which can be a computer or mobile device (106). This functionality can be encapsulated inside either pressure monitor or processor unit in alternative embodiments.
   c) At least one medical imaging device, for example an ultrasound probe (107) with wired or wireless digital output permitting to stream the output data into the processor unit (106). The said probe is pointed to the same cardiac region where the sensor (101) is located.
   d) Processor unit, preferably a computer or a mobile device equipped with hardware and software permitting synchronization, recording, storage and processing of input data from medical imaging device and medical pressure monitor. The said processor unit performs a simultaneous recording 30-60 seconds from both medical imaging device and pressure measurement monitor.
2) For the General (or post-calibration) Usage Procedure (FIG. 2)
   a) At least one medical imaging device, for example an ultrasound probe (201) with wired or wireless digital output permitting to stream the output data into the processor unit (203).
   b) Processor unit (203), which can be a computer or a mobile device equipped with hardware and software permitting recording, storage and processing of input data from medical imaging device.
3) Optionally the hardware system of the embodiment may include a remote cloud or other specialized server (110, 206, 302, and 402), operation of which permits both processor units (106, 203) to store, retrieve and exchange data if internet connection is available, but it is generally possible to transfer the data by other means, directly between the devices or by physical medium.
4) During the Calibration Procedure, the software, positioned either on the processor unit (106) or on remote cloud or other specialized server (110) processes the recorded data by using the algorithm described in items 7)-9), creates and stores a calculation model (303) for calculation of subsequent pressure results from medical imaging device unit recordings of the previously calibrated user. This model is transferred directly or via the server to the processor unit.
5) During the General Usage Procedure (FIG. 3, 4) the said calculation model (303) is accessed either locally or on server (302) by processor unit to calculate the user's intracardiac pressure according to the data recorded by the imaging device (202).
6) In absence of previous calibration model for the specific user (FIG. 4) the user performs the recording procedure (401), sending the data to remote cloud or other specialized server (402), which retrieves the stored calibration models (403) of other users with similar physiological profile (age, weight, height, diagnoses, etc.) and uses machine learning to calculate the result according to said models. In this case use of a server is required.
7) The data recorded during the Calibration Procedure is processed as follows:
   a) The data received from the imaging device decoded into image frames (FIG. 5). The frames may be 2 or 3-dimensional, depending on the imaging technology.
   b) Each frame (FIG. 6) is marked with a time stamp at which it was recorded. In this example the difference between frames (601) and (602) is about 0.5 second.
   c) T-Image {Tᵢ}_{i=1, . . . N} (FIG. 7) (701) is formed from the data. It comprises an array of imaging data (702) linked to an appropriate time axis (703) and synchronized with the measured pressure values P_{i, i=1, . . . N} obtained by the said clinical catheterization pressure measurement monitor in oscillating traceable region(s) which can be for example the Right/Left Atrium, Right/Left Ventricle, or other major blood vessels in heart vicinity.
   d) The software determines the physiological features that can be detected in the imaging data (FIG. 8). On the two frames (801, 802) separated in FIG. 8 by 0.5 second, the software locates the heart (803, 804), and the positions and contours of oscillating traceable region(s) as the Right Atrium (805), Left Atrium (806), Right Ventricle (807) and Left Ventricle (808).
   e) If, for example, the sensor during the calibration was located in the Left Ventricle (FIG. 9) of the user's heart, the software separates the Left Ventricle (903, 904) on each frame (901, 902).
   f) The software then converts the detected contour (903, 904) into a set of coordinates {xⱼ}, representing the form of the target region. This coordinate set may contain any number of point coordinates, depending on the processing power available to the software and image resolution of the imaging device, with at least two point coordinates (the depths where the target region begins and where it ends) required to make a pressure assessment.
   g) The software then compares (FIG. 10) the changes in coordinate sets in each frame (1002) to the changes in pressure (1001) measured at the same time, and builds a calculation model by fitting the measured pressure to a functional shape P(tᵢ)=P(tᵢ, {xⱼ}_{j=1, . . . M}ⁱ⊂Tᵢ), where P(tᵢ) is the measured pressure at time tᵢ of each imaging frame.
   h) The calculation model is stored in the processor unit and later transferred to user's processor unit using cloud or other specialized server or directly or by any other means.
8) For example, the detection of the physiological features as described above in 7(d) may be performed, among other possible methods, by using Time Derivative streams {Tᵢ}′_{i=1, . . . N}, {Tᵢ}″_{i=1, . . . N}, of the T-image, where:
   a) For the first derivative {Tᵢ}′_{i=1, . . . N} each frame represents the difference in brightness or color channel values between two subsequent frames of the original T-image. The result represents the speed of movement of the detected tissue from frame to frame.
   b) For the second derivative {Tᵢ}″_{i=1, . . . N} each frame represents the difference between two subsequent frames of the first derivative. The result represents the acceleration of movement of the detected tissue from frame to frame.
   c) In a similar manner it is possible to produce and utilize further derivative streams.
   d) Averaging the derivative streams along the time scale (thus turning the stream into a single image, where each pixel is an average of the respective pixels with the same coordinates on all frames of the T-image) enables to detect regions where the tissue movement is repeating and by marking them the software can identify the regions of interest or advice the user to move the imaging device to obtain a more correct view of the heart.
9) Additionally to the method described above in item 7) a simplified method of processing may be used which comprises
   a) Conversion of T-Image into a Characteristic Image (FIG. 11). Each frame {Tᵢ}_{i=1, . . . N} of imaging data (1101) is compressed (1102) using averaging or other invariant method to a single column (1103). In the same manner, the T-Image (1104) containing series of frames and a time axis (1105) is compressed to a Characteristic Image (1106) with number of columns identical to number of frames and the same time axis.
   b) In a process similar to items 7(d)-7(f) the software determines the boundaries of the target images on the compressed columns, determining the minimum required two (the depths where the target region begins and where it ends), although using various image processing techniques more information on the region's form may be determined, and creates the calculation model in similar way to the item 7(g). The similarity (FIG. 12) between the synchronized pressure measurements of LA (1202) and LV (1203) as received from the pressure monitor (105) and the respective calculated pressure curves LA (1204) and LV (1205) in the Characteristic Image (1201) is clearly identifiable. This method enables to greatly increase the speed of processing and reduces the computational power requirements while maintaining enough accuracy given the images are recorded from a similar angle to the calibration.
10) During General Usage Procedure, the user
   a) Uses the imaging device in the same manner as during the calibration to make a recording for a of a preset time period while pointing at the same region that was recorded during the calibration. The region and angle to which the imaging device is directed are marked during the calibration procedure and made available to the user via graphical user interface (GUI).
   b) The imaging device transmits by wired or wireless connection the image sequence to the processor unit.
   c) The processor unit retrieves the user's model (if the user had undergone personal calibration) from internal memory/cloud service/storage medium.
   d) If the user did not undergo the personal calibration procedure, the processor unit transmits the user's data such as height, weight, diagnosis, stored in similar way to the cloud service or specialized server. The cloud service or specialized server returns a model created using machine learning based on the database of calibrated models for users with similar characteristics.
   e) The software on the processor unit then processes the images obtained from the imaging device during the recording in a manner similar to items 7(d)-7(f) or 9), obtaining the coordinate set {xⱼ}.
   f) Using the said coordinate set {xⱼ} and the model P(tᵢ)=P(tᵢ, {xⱼ}_{j=1, . . . M}ⁱ⊂Tᵢ), the software calculates its assessment of pressure and displays it to the user.
   g) The software may optionally transmit this assessment to the server, display it to the doctor using doctor's dedicated system and/or show system alerts if it detects abnormal values or patterns.
11) The other real time characteristics which can be measured or estimated using the described method include but not limited to: Left Atrial Pressure (LAP), Right Atrial Pressure (RAP), Left Ventricular Pressure Rise dP/dt_{max,L} (LVPR), Right Ventricular Pressure Rise dP/dt_{max,R} (RVPR), Pulmonary Artery Pressure (PAP), Pulmonary Capillary Wedge Pressure (PCWP), Left Ventricular Systolic Pressure (LVSP), and Right Ventricular Systolic Pressure (RVSP), LVEDP (Left Ventricular End-Diastolic Pressure) and RVEDP (Right Ventricular End-Diastolic Pressure).
12) The described method is implemented as a software executed on processor units with at least the following capabilities:
   a) Both processor units used during the calibration procedure and during the general usage procedure may be any computing devices restricted only by ability to run processing software and provide necessary data connections and user interfaces.
   b) For the processor unit during calibration the software will:
      i) Provide a real-time connection for data retrieval from
         (1) Medical imaging device
         (2) Pressure sensor(s) an/or a Pressure Monitor
      ii) Display the images and pressure data acquired from said devices, including image stream for targeting the region of interest.
      iii) Provide assistance in targeting for the user on GUI.
      iv) Perform a synchronized data recording from medical imaging device and pressure monitor.
      v) Store and transmit the acquired data to readable medium, other devices or cloud server.
      vi) Perform analysis and fitting of pressure calculation model based on acquired data.
   c) For Processor unit during general usage procedure the software will:
      i) Provide a real-time connection for data retrieval from medical imaging device.
      ii) Display the images and pressure data acquired from said device, including image stream for targeting the region of interest.
      iii) Provide assistance in targeting for the user on GUI.
      iv) Perform a recording of a preset length from medical imaging device.
      v) Store and transmit the acquired data to readable medium, other devices or cloud server.
      vi) Store and retrieve the calculation mode from internal memory, readable medium or the cloud server.
      vii) Perform a calculation of pressure based on calibration model and acquired data.
      viii) Detect anomalies and display alerts on user interface.
   d) For cloud or dedicated server system:
      i) Store and retrieve user data, recordings and calculation models.
      ii) Provide user data to respective medical service providers including recording results, pressure trends, etc.
      iii) Collect and provide cumulative models from users with similar characteristics for users without personal calibration.
      iv) Perform all analysis and calculations similar to processor units (12(b), 12(c)).
      v) Provide user interfaces for medical service providers and users.
      vi) Provide connection interfaces for processor units.

The present invention has been described using a non-limiting detailed description of various embodiments and examples thereof. It should be appreciated that the present invention is not limited by the above-described examples and that one ordinarily skilled in the art can make changes and modifications without deviation from the scope of the invention as will be defined below in the appended claims.

Within the scope of invention as defined by the appended claims the medical imaging device can be combined with the processor unit into a single device.

It should also be appreciated that features disclosed in the foregoing description, and/or in the foregoing drawings and/or following claims both separately and in any combination thereof, be material for realizing the present invention in diverse forms thereof. When used in the following claims, the terms “comprise”, “include”, “have” and their conjugates mean, “including but not limited to”.

The present invention has been described above with reference to specific examples. However, other embodiments than the above described are equally possible within the scope of the invention. Different method steps than those described above, performing the method by hardware or software, may be provided within the scope of the invention. The different features and steps of the invention may be combined in other combinations than those described. The scope of the invention is only limited by the appended patent claims.

1. A method for non-invasive measurement of intracardiac pressure utilizing
a medical imaging device capable of highlighting cardiovascular structures, such as heart chambers and major blood vessels connected to the heart of a user (heart vicinity) and configured to receive commands and transmit the obtained image stream {Jᵢ}_{i=1, . . . N} (where i is the index of the image and N is the number of images in the stream) in real time through a network; and
a processor unit configured to calculate dynamically changing intracardiac pressures of a user and being capable
i. to communicate with said imaging device and equipped with software providing communication with the said imaging device and the network,
ii. to process and display the obtained data on graphical user interface (GUI),
iii. to store the calculation result either locally or using internet or cloud services and display the said result to the user via GUI,
and configured to
record an initial image record the image stream {Jᵢ}_{i=1, . . . N} of cardiovascular movement within a user's heart vicinity from the said imaging device,
process the image stream {Jᵢ}_{i=1, . . . N} generating the corresponding T-image {Tᵢ}_{i=1, . . . N} defined as a chronological union of said image stream {Jᵢ}_{i=1, . . . N} data with corresponding time stamps,
process the said {Tᵢ}_{i=1, . . . N} to determine oscillating traceable regions corresponding to the said cardiovascular structures and record the changes over time of the shapes of the said cardiovascular structures, where an oscillating traceable region is defined as a region, appearing on most images comprising the said T-Image {Tᵢ}_{i=1, . . . N} and corresponding to the said cardiovascular structures where the pressure is calculated,
produce pressure Pᵢ changes time series {ΔP(tᵢ)}_{i=1, . . . N} from the said shape changes of oscillating traceable regions;
the said method comprising of the following method steps, where:
the said imaging device is connected to the processor unit,
the said imaging device is pointed towards the cardiac region, activated and synchronized with the processor unit,
the said imaging device transmits the said image stream {Jᵢ}_{i=1, . . . N} to the processor unit which stores the said image stream generating the corresponding T-image {Tᵢ}_{i=1, . . . N},
on obtaining the said T-image {Tᵢ}_{i=1, . . . N} from the said imaging device, the said processor unit loads and uses the mathematical model to calculate intracardiac pressure from the said image stream {Jᵢ}_{i=1, . . . N} obtained from the said imaging device,
where the mathematical model comprises the method steps of creating and using:
the Time Derivative Streams {Tᵢ}′_{i=1, . . . N}, {Tᵢ}′_{i=1, . . . N}, . . . of the said T-image {Tᵢ}_{i=1, . . . N}, which:
are created as pixel-wise finite differences Tᵢ−Tᵢ₋₁ of the brightness if the said T-image is in greyscale or a value of respective color channel in case the said T-image is in color,
are subsequently averaged along the time scale (mean{Tᵢ}′_{i=1, . . . N}, mean {Tᵢ}″_{i=1, . . . N}, . . . ), and
are permitting to identify the size and position of the oscillating traceable regions by tracking the patterns of repeating changes, which appear as consistent spots of high brightness value on the said time derivative streams; and
the Characteristic Image, which:
is defined as a chronological union of the averages of the rows or other invariants of the said T-image {Tᵢ}_{i=1, . . . N} across each given depth, in the way that the first pixel-column I₁(i=1) of the Characteristic Image contains the averages over the rows or other invariants of the first image in time, the second pixel-column I₂(i=2) of the Characteristic Image contains the averages over the rows or other invariants of the second image in time, and finally the last pixel-column I_{N}(i=N) of the Characteristic Image contains the averages over the rows or other invariants of the last image in time in the series, where
the invariants in the Characteristic Image can be:
averages of the columns,
vertical or horizontal average gradients,
singular values or eigenvalues of each image packed into the Characteristic image as one matrix,
Fourier, Wavelet or other generalized decomposition images of the Characteristic Image;
is permitting to visually represent and rapidly determine the changes of the size and boundaries of the targeted oscillating traceable region over the said T-image;
and where the method steps for the assessing of intracardiac pressure changes time series {ΔP(tᵢ)}_{i=1, . . . N} further include:
estimating the size of said target oscillating traceable region(s) from each frame of the said T-image {Tᵢ}_{i=1, . . . N},
creating a set of coordinate parameters {xⱼ}_{j=1, . . . M} (where j is an index of the coordinate and M is the total number of the coordinate parameters) representing the said oscillating traceable region size and position at each time corresponding to each frame Tᵢ of the said T-image {Tᵢ}_{i=1, . . . N},
estimating the pressure changes {ΔP(tᵢ)}_{i=1, . . . N} by a functional pressure shape Pᵢ=P(tᵢ, {xⱼ}_{j=1, . . . M}ⁱ⊂Tᵢ), provided a given mean pressure level and functional shapes from the stored mathematical models for the current user or other users with similar physiological data are available.

2. The method of claim 1 further includes
creation of the mathematical model for measurement of intracardiac pressure from imaging data obtained from said imaging device during Calibration Procedure, which comprises of:
utilization of a said processor unit and a medical imaging device from claim 1 synchronized with a standard clinical catheterization pressure measurement monitor configured to receive pressure values from pressure sensors located inside cardiovascular structures such as heart chambers and major blood vessels connected to the heart and transmit the said pressure values to the said processor unit from claim 1 during a clinical catheterization, wherein the processor unit communicates with said imaging device, said clinical catheterization pressure measurement monitor, and is equipped with software providing communications with the said devices, and further processes the-obtained data and displays the said data on graphical user interface (GUI) and is configured to control said clinical catheterization pressure measurement monitor and said imaging device in the way that it is capable to:
synchronize the T-Image {Tᵢ}_{i=1, . . . N} obtained by the said imaging device with the measured pressure values P_{i, i=1, . . . N} obtained by the said clinical catheterization pressure measurement monitor in the said oscillating traceable region(s),
fit absolute pressure values corresponding to pressure changes time series {ΔP(tᵢ)}_{i=1, . . . N} from claim 1 to the measured pressure values P_{i, i=1, . . . N} obtained from the said clinical catheterization pressure measurement monitor, through producing the parameters of a mathematical model defined by the functional pressure shape Pᵢ=P(tᵢ, {xⱼ}_{j=1, . . . M}ⁱ⊂Tᵢ), from claim 1 corresponding to the change of the shape of each said oscillating traceable region,
store the said parameters of the mathematical model obtained from the above fit procedure performed during clinical catheterization;
assessment of the size of said target oscillating traceable region(s) from each frame of the said T-image {Tᵢ}_{i=1, . . . N},
creation of a set of coordinate parameters {xⱼ}_{j=1, . . . M} (where j is an index of the coordinate and M is the total number of the coordinate parameters) representing the said oscillating traceable region size and position at each time corresponding to each frame Tᵢ of the said T-image {Tᵢ}_{i=1, . . . N},
fitting of the said measured pressure Pᵢ to a functional shape Pᵢ=P(tᵢ, {xⱼ}_{j=1, . . . M}ⁱ⊂Tᵢ), and
usage of the said mathematical model for measurement of intracardiac pressure from imaging data obtained from said imaging device, which comprises:
utilization of a said processor unit and a medical imaging device from claim 1 wherein the processor unit is capable to communicate with said imaging device and equipped with software providing communications with the said device, and further capable to process the-obtained data and display the said data on graphical user interface (GUI) and is configured to control said imaging device in the way that it is capable to:
receive and store the T-Image {Tᵢ}_{i=1, . . . N} obtained by the said imaging device,
read the said parameters of the mathematical model obtained from the above fit procedure performed during clinical catheterization,
re-use the said parameters of the mathematical model on subsequent recordings of the said oscillating traceable regions with said imaging device connected to the said processor unit to calculate absolute pressure values P_{i, i=1, . . . N} from the said pressure changes time series {ΔP(tᵢ)}_{i=1, . . . N};
assessment of the size of said target oscillating traceable region(s) from each frame of the said T-image {Tᵢ}_{i=1, . . . N} obtained during the usage recording,
creation of a set of coordinate parameters {xⱼ}_{j=1, . . . M} representing the said oscillating traceable region size, form and position at each time corresponding to each frame Tᵢ of the said T-image {Tᵢ}_{i=1, . . . N},
comparison of the said set of coordinate parameters against the said functional shape and produce an estimate of real-time pressure value time series P(tᵢ)=P(tᵢ, {xⱼ}_{j=1, . . . M}ⁱ⊂Tᵢ), and
estimation of the pressure changes {ΔP(tᵢ)}_{i=1, . . . N} in case of the absence of the said calibration procedure for the current user, and estimation of the pressure values P(tᵢ) in case of absence of the said calibration procedure for the current user using the functional shapes from the stored mathematical models of other users with similar physiological data if available.

3. A software including code segments corresponding to method steps from claim 1.

4. A software including code segments corresponding to method steps from claim 2.
